(19) United States
(12) Patent Application Publication
(10) Pub. No.: US 2004/0235904 A1
(43) Pub. Date: Nov. 25, 2004

(54) Crystalline and amorphous solids of pantoprazole and processes for their preparation

(76) Inventors: Nina Finkelstein, Herzliya (IL); Barnaba Krochmal, Jerusalem (IL); Shlomit Wizel, Petah Tiqva (IL)

Correspondence Address: KENYON & KENYON, ONE BROADWAY, NEW YORK, NY, 10004, US

(21) Appl. No.: 10/799,376

(22) Filed: Mar. 12, 2004

Related U.S. Application Data
(60) Provisional application No. 60/464,358, filed on Apr. 22, 2003.
(60) Provisional application No. 60/453,836, filed on Mar. 12, 2003.

(51) Int. Cl. C 07D 4 3/02; A61K 31/4439

(52) U.S. Cl. 514/338; 546/273.7

(57) ABSTRACT

The present invention relates to a polymorphic form (Form I) of pantoprazole and a processes of making same. Form I has a PXRD pattern with characteristic peaks at 6.6, 13.2, 13.7, 15.7, 23.1, and 23.4±0.2 °2θ and a FTIR spectrum with characteristic bands at 1385, 1264, 1244, 1180, and 1027 at cm⁻¹. The process of making Form I includes crystallizing pantaoprazole or forming slurry from amorphous pantoprazole. The present invention also relates to another polymorphic form (Form II) of pantoprazole and a process of making the same. Form II has a PXRD pattern with characteristic peaks at 5.8, 7.5, 9.3, 15.0, 22.0, and 22.6±0.2 °2θ and a FTIR spectrum with characteristic bands at 3195, 1196, and 1584 at cm⁻¹. The process of making Form II includes forming a slurry from amorphous pantoprazole.

### CROSS-REFERENCE TO RELATED APPLICATIONS

This application claims priority to U.S. Provisional Application No. 60/464,358 filed on Apr. 22, 2003 and U.S. Provisional Application No. 60/453,836 filed on Mar. 12, 2003.

### FIELD OF THE INVENTION

The present invention relates to the solid state properties of pantoprazole, a gastric acid secretion inhibitor.

### BACKGROUND OF THE INVENTION

Pantoprazole is a gastric acid secretion inhibitor. The systematic chemical name of pantoprazole is 5-(difluoromethoxy)-2-[[(3,4-dimethoxy-2-pyridinyl)methyl]sulfinyl]-1H-benzimidazole and its molecular structure is represented by formula (I).

(I)

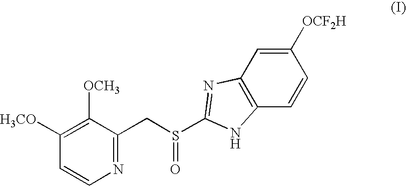

U.S. Pat. No. 4,758,579 discloses that pantoprazole and many other fluoroalkoxy substituted benzimidazoles are gastric acid secretion inhibitors. The '579 patent states that pantoprazole can be prepared by oxidation of the sulfide analog with meta-chloroperbenzoic acid (MCPBA) by following a procedure described in Example 2 of the '579 patent. According to Example 2, the oxidation is conducted in dichloromethane. The reaction mixture is quenched with sodium thiosulfate and sodium carbonate. The product is extracted from the aqueous phases with dichloromethane, washed with sodium thiosulfate, dried over magnesium sulfate and concentrated to low volume under vacuum giving solid pantoprazole after addition of diisopropyl ether.

The pharmaceutical product Protonix® is marketed in the United States by Wyeth-Ayerst for short term treatment of erosive oesophagitis caused by gastric reflux disease. According to the package insert for Protonix®, the product contains pantoprazole sodium sesquihydrate.

The present invention relates to the solid state physical properties of pantoprazole. These properties can be influenced by controlling the conditions under which pantoprazole is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state 13C NMR spectrometry and infrared spectrometry.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. New polymorphic forms of pantoprazole have now been discovered.

### SUMMARY OF THE INVENTION

The present invention provides new crystalline forms of pantoprazole and processes of forming such new crystalline forms. The present invention also provides a new amorphous form of pantoprazole as well as a process for forming such new amorphous form. The present invention additionally provides a salt of pantoprazole formed from amorphous pantoprazole or the crystalline forms of pantoprazole according to the present invention.

In one aspect, the present invention provides crystalline solid pantoprazole Form I characterized by a PXRD pattern having peaks at 6.6, 13.2, 13.7, 15.7, 23.1, and 23.4±0.2 °2θ. Form I can be prepared by dissolving pantoprazole in a solvent, precipitating crystals of the pantoprazole Form I from the solution, and separating the crystals from the solvent. Form I can also be prepared by forming a slurry of amorphous pantoprazole with a diluent, maintaining the slurry for a period of time sufficient to convert the amorphous pantoprazole to pantoprazole Form I and then separating the pantoprazole Form I from the diluent.

In another aspect, the present invention provides crystalline solid pantoprazole Form II characterized by a PXRD pattern having peaks at 5.8, 7.5, 9.3, 15.0, 22.0, and 22.6±0.2 °2θ. Form II can be prepared by forming a slurry of amorphous pantoprazole in a diluent, maintaining the mixture for a period of time sufficient to convert the amorphous pantoprazole to Form II and separating the diluent.

In yet another aspect, the present invention provides amorphous pantoprazole. Amorphous pantoprazole can be prepared by partitioning pantoprazole between the organic and aqueous phase of a biphasic mixture of a water-immiscible organic liquid and water, adding acid (such as an organic acid) to the mixture, separating the organic phase and the water, and recovering the amorphous pantoprazole from the organic phase.

### BRIEF DESCRIPTION OF THE FIGURES

FIG. 1 is a representative PXRD pattern of pantoprazole Form I.

FIG. 2 is a representative FTIR pattern of pantoprazole Form I.

FIG. 3 is a representative PXRD pattern of pantoprazole Form II.

FIG. 4 is a representative FTIR pattern of pantoprazole Form II.

### DETAILED DESCRIPTION OF THE INVENTION

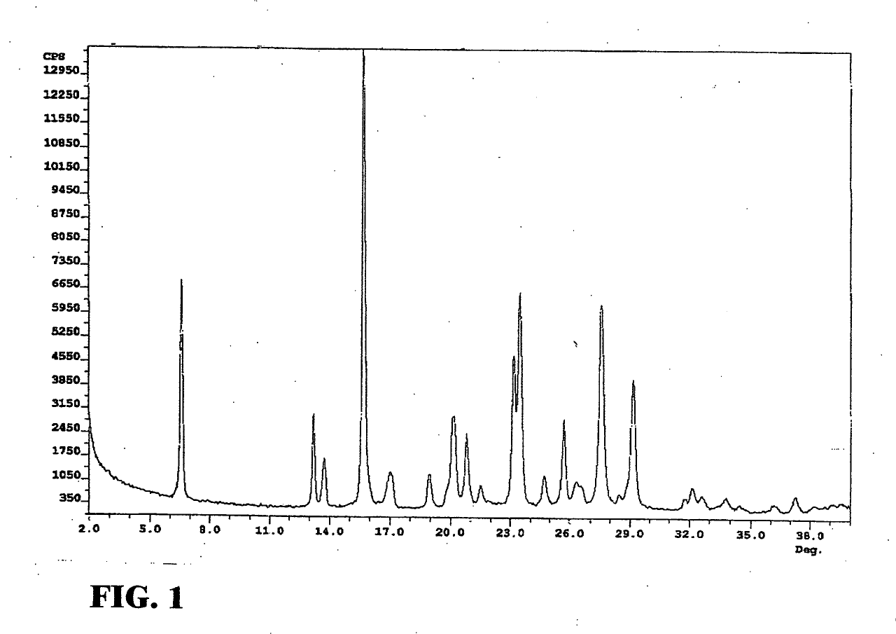

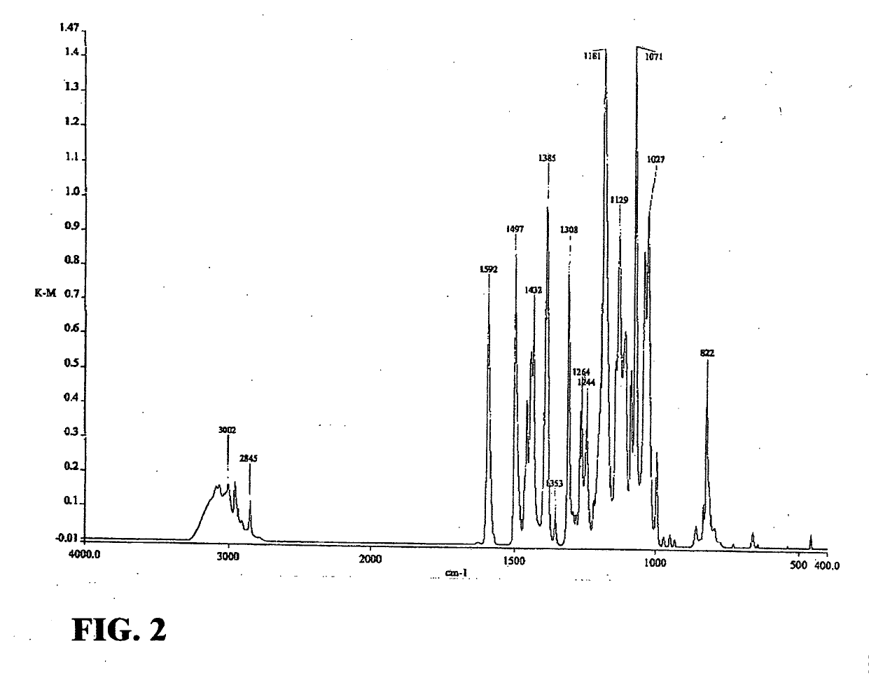

In one aspect, the present invention provides a solid crystalline form of pantoprazole that has been denominated “Form I.” A representative example of the powder X-ray diffraction (PXRD) pattern of Form I is provided in FIG. 1, where characteristic peaks occur at 6.6, 13.2, 13.7, 15.7, 23.1, and 23.4±0.2 °2θ. Additional peaks occur at 20.1, 20.9, 25.9, 27.5 and 29.1±0.2 °2θ. A representative example of the Fourier Transform Infrared (FTIR) spectrum of Form I is provided in FIG. 2, where characteristic bands occur at 1385, 1264, 1244, 1180, and 1027 at cm⁻¹.

Pantoprazole Form I can be prepared by dissolving pantoprazole in a solvent such as ethanol, n-propanol or acetone and by heating to about 55°, followed by cooling and stirring at ambient temperature for 1 hour. The solid is then separated out from the solvent by, for example, filtering or decanting. Preferably, the solid is separated out by filtering.

Alternatively, Form I can be prepared by a “slurry method.” Slurry may be defined as a heterogeneous mixture or undissolved particles in a liquid. In particular, Form I can be prepared from a starting material of amorphous pantoprazole in a slurry with a diluent selected from the group consisting of ethanol, acetone, n-propanol, ethyl acetate, tetrahydrofuran, sec-butanol, dimethylcarbonate, mixtures of methyl tert butyl (MTBE) and water (such as MTBE-water (5%)), mixtures of dimethylcarbonate and water (such as dimethylcarbonate-water (5%)), mixtures of sec-butanol and water (such as sec-butanol-water (5%)), and mixtures of dichloromethane and water (such as dichloromethane and water (5%)). The method further comprises maintaining contact of the amorphous pantoprazole with the diluent for at least 12 hours and then separating the solid from the diluent, such as by filtering or decanting. Preferably, the solid is separated from the diluent by filtering.

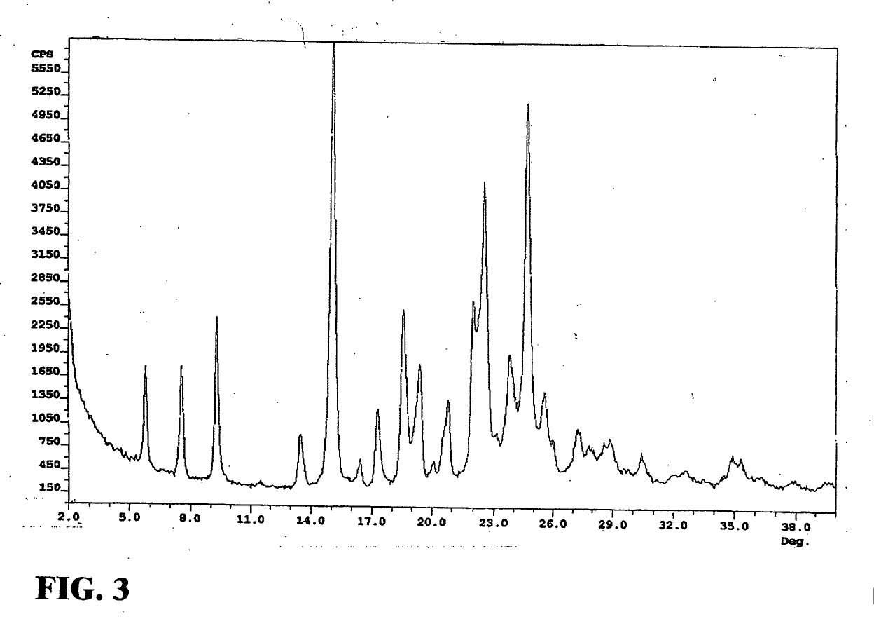

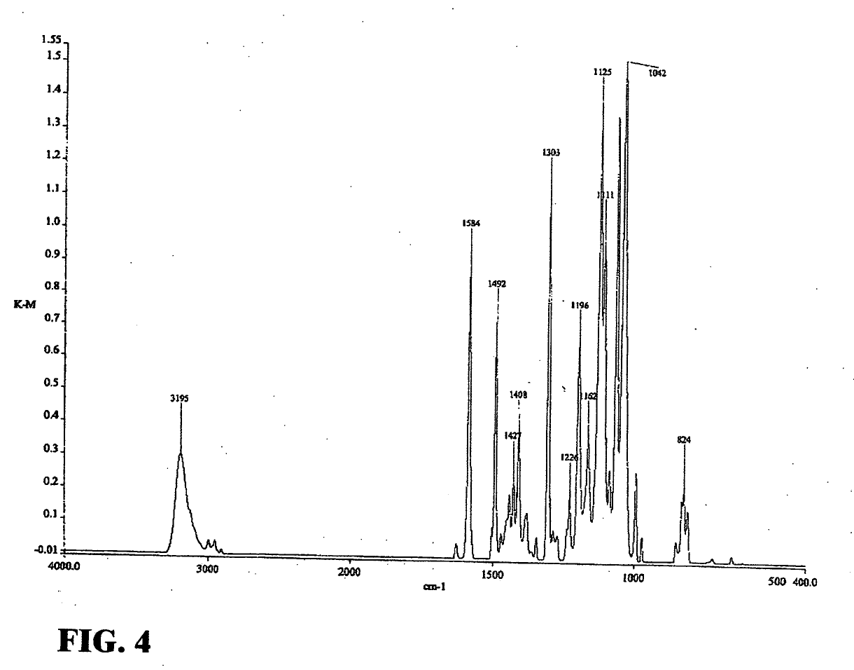

In another aspect, the present invention provides a solid crystalline form of pantoprazole that has been denominated “Form II.” Form II can be differentiated from Form I by its powder X-ray diffraction pattern. A representative example of the PXRD pattern of Form II is provided in FIG. 3, where characteristic peaks occur at 5.8, 7.5, 9.3, 15.0, 22.0, and 22.6±0.2 °2θ. Additional peaks occur at 17.3, 18.6, 19.4, 20.8, 24.0, 24.8, and 25.5±0.2 °2θ. Form II can also be differentiated from Form I by its Fourier Transform Infrared (FTIR) spectrum. A representative example of the FTIR spectrum of Form II is provided in FIG. 4, where characteristic bands can be found at 3195, 1196, and 1584 at cm⁻¹. Form II according to the present invention has a melting endotherm at about 143° C. to about 146° C. by DSC analyses.

Form II also can be prepared by a “slurry method.” In particular, Form II can be prepared from a starting material of amorphous pantoprazole by forming a slurry of amorphous pantoprazole in a diluent selected from the group consisting of diethyl ether, and tert-butyl methyl ether (MTBE). The method further comprises maintaining contact of the amorphous pantoprazole with the diluent for preferably 24 hours (and in the case of MTBE for 48 hours) and then separating the solid from the diluent, such as by filtering or decanting. Preferably, the solid is separated from the diluent by filtering.

In another aspect, a mixture of Form I and Form II can be prepared by a “slurry method.” In particular, the mixture of Form I and Form II can be prepared from a starting material of amorphous pantoprazole by forming a slurry of amorphous pantoprazole in a diluent such as mixtures of toluene and water (such as toluene-water 5%)) and MTBE. The method further comprises maintaining contact of the amorphous pantoprazole with the diluent for preferably 24 hours (and in the case of MTBE for 48 hours) and then separating the solid from the diluent, such as by filtering or decanting. Preferably, the solid is separated from the diluent by filtering.

PXRD analysis for both Form I and II, the results of which are depicted in FIGS. 1 and 3 was performed on a Scintag X-ray powder diffractometer model X′TRA, Cu-tube, solid state detector. A round standard aluminum sample holder with round zero background quartz plate was used. Scanning parameters were: Range 2-40 °2θ:continuous scan, Rate: 3°/minute. DSC analysis was performed on DSC831e, Mettler Toledo, Sample weight: 3-5 mg, Heating rate: 10° C./minute, Number of holes in the crucible: 3. FTIR spectra were performed on Perkin-Elmer spectrum One Spectrometer, Diffuse Reflectance Technique. The sample was finely ground with potassium bromide, and the spectrum was recorded using potassium bromide background in a diffused reflectance accessory.

In yet another aspect, the present invention provides novel amorphous pantoprazole in free base form that produces a featureless PXRD pattern. According to a process of the present invention, a free base of pantoprazole is partitioned between the organic and aqueous phases of a biphasic mixture of a halogenated hydrocarbon or other water-immiscible organic liquid and water at room temperature. A preferred organic liquid is dichloromethane. Pantoprazole can be added to the mixture as a free base, such as Forms I and II or as a salt, such as pantoprazole sodium or pantoprazole potassium. An especially preferred starting material is pantoprazole sodium. According to this process, the biphasic mixture should be agitated, such as by stirring, to ensure efficient exchange between the two phases. Acid, preferably an organic acid such as acetic acid is then added with one molar equivalent thereof with respect to the pantoprazole being a generally sufficient amount. After completion of the addition of the acid, agitation is ceased and the phases are separated. Amorphous pantoprazole free base is then recovered from the organic phase by conventional means, such as by evaporation of the organic liquid at 30° C.

The pantoprazole free base forms and amorphous pantoprazole can be converted to a salt of pantoprazole, such as pantoprazole sodium by known methods. In addition, the sodium salt of pantoprazole can be formed by methods described in our commonly assigned co-pending U.S. patent application Ser. No. 10/739,272, which is hereby incorporated by reference in its entirety and in particular for its teachings regarding the preparation of pantoprazole sodium from pantoprazole free base, which teachings are exemplified in Examples 11, 18, 26, 44, 61-63 and 64. For example, sodium hydroxide and any of pantoprazole Form I, Form II, or amorphous pantoprazole may be dissolved in a solvent, such as butanol, propanol, methanol, ethanol, and then a salt of pantoprazole may be precipitated from the solution. In one exemplary process, pantoprazole of Forms I, II, or amorphous pantoprazole is stirred with ethyl acetate and aqueous sodium hydroxide and the mixture is stirred overnight at room temperature. The pantoprazole is then isolated. When pantoprazole Form I is used, the pantoprazole sodium is sesquihydrate.

Forms I and II, amorphous pantoprazole, and salts of pantoprazole are useful as gastric acid secretion inhibitors. For this purpose, they can be formulated into a variety of compositions for administration to humans and animals. Accordingly, the present invention further provides pharmaceutical compositions that contain Form I, Form II, amorphous pantoprazole, salts of pantoprazole or mixtures thereof with each other or with other forms of pantoprazole. In addition to the active ingredient(s), the pharmaceutical compositions of the present invention can contain one or more excipients. Excipients are added to the composition for a variety of purposes.

For example, diluents increase the bulk of a solid pharmaceutical composition and can make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet can include excipients whose functions include helping to bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach can be increased by the addition of a disintegrant to the composition. Disintegrants include for example alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

When a dosage form such as a tablet is made by compaction of a powdered composition, the composition is subjected to pressure from punches and a die. Some excipients and active ingredients have a tendency to adhere to the surfaces of the punches and die, which can cause the product to have pitting and other surface irregularities. A lubricant can be added to the composition to reduce adhesion and ease release of the product form the die. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fiunarate, stearic acid, talc and zinc stearate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, pantoprazole Forms I and II, amorphous pantoprazole, salts of pantoprazole or mixtures thereof along with any other solid excipients are dissolved or suspended in a liquid carrier such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain a viscosity enhancing agent to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at levels safe for ingestion to improve storage stability.

A liquid composition according to the present invention can also contain a buffer such as gluconic acid, lactic acid, citric acid or acetic acid, sodium guconate, sodium lactate, sodium citrate or sodium acetate.

All above-described excipients and other excipients known by one of skill in the art are contemplated by the present invention. Selection of excipients and the amounts to use can be readily determined by the formulation scientist based upon experience and consideration of standard procedures and reference works in the field.

The solid compositions of the present invention include powders, granulates, aggregates and compacted compositions. The dosages include dosages suitable for oral, bucal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

Dosage forms include solid dosage forms like tablets, powders, capsules, suppositories, sachets, troches and lozenges as well as liquid syrups, suspensions and elixirs. For example, the dosage form of the present invention may be a capsule containing the composition, preferably a powdered or granulated solid composition of the invention, within either a hard or a soft shell. The shell can be made from gelatin and optionally contain a plasticizer such as glycerin and sorbitol, and an opacifying agent or colorant.

The active ingredient and excipients can be formulated into compositions and dosage forms according to methods known in the art. For example, a composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can also be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

Capsules, tablets and lozenges and other unit dosage forms preferably contain a dosage level of about 10 to about 100 mg of pantoprazole Form I or II, amorphous pantoprazole, a salt of pantoprazole, or mixtures thereof, more preferably about 45 mg. Other dosages may also be administered depending on the need. In preferred embodiment, a pharmaceutical composition according to the present invention is a delayed-release tablet for oral administration that contains about 40 mg to about 45 mg of Form I or II, amorphous pantoprazole, a salt of pantoprazole, or mixtures thereof. Excipients include anhydrous sodium carbonate NF, mannitol USP, crospovidone NF, povidone USP, calcium stearate NF, hydroxypropyl methylcellulose USP, titanium dioxide USP, yellow iron oxide NF, propylene glycol USP, methacrylic acid copolymer NF, polysorbate 80 NF, sodium lauryl sulfate NF, and triethyl citrate NF.

### EXAMPLES

### Examples 1-3

### Preparation of Pantoprazole Form I

Pantoprazole was dissolved in a suitable solvent by heating to about 55° C., followed by cooling and stirring at ambient temperature for one hour. The solid was filtered and analyzed by PXRD as a wet product and after drying in a vacuum at 50° C. overnight.

### Examples 4-18

### Preparation of Pantoprazole Form I and/or Form II by the Slurrying Method

Pantoprazole amorphous form was slurried in a suitable solvent at ambient temperature. The solid was filtered and analyzed by PXRD as wet product and after drying in vacuum at 50° C. overnight.

| | | | Pantoprazole/ | | Crystal Form | Crystal Form |
|---|---|---|---|---|---|---|
| | | | Solvent Ratio | Time of | of Wet | of Dry |
| Ex | Solvent | Procedure | (g)/(ml) | Slurry | Sample | Sample |
| 1 | Ethanol | Crystallization | 3 g/10 ml | | I | I |
| 2 | n-propanol | Crystallization | 3 g/10 ml | | I | I |
| 3 | Acetone | Crystallization | 1 g/5 ml | | I | I |
| 4 | Ethanol | Slurry | 1 g/4 ml | 12 h | I | I |
| 5 | n-propanol | Slurry | 1 g/5 ml | 14 h | I | I |
| 6 | Ethyl Acetate | Slurry | 1 g/5 ml | 12 h | I | I |
| 7 | Tetrahydrofuran | Slurry | 1 g/4 ml | 24 h | I | I |
| 8 | Sec-butanol | Slurry | 1 g/5 ml | 24 h | I | I |
| 9 | Dimethylcarbonate | Slurry | 1 g/5 ml | 24 h | I | I |
| 10 | MTBE-water (5%) | Slurry | 1 g/5 ml-0.25 ml | 24 h | I | I |
| 11 | Dimethylcarbonate- | Slurry | 1 g/5 ml-0.25 ml | 24 h | I | I |
| | water (5%) | | | | | |
| 12 | Sec-butanol-water | Slurry | 1 g/5 ml-0.25 ml | 24 h | I | I |
| | (5%) | | | | | |
| 13 | Dichloramethane- | Slurry | 1 g/5 ml-0.25 ml | 24 h | — | I |
| | water (5%) | | | | | |
| 14 | Toluene-water (5%) | Slurry | 1 g/5 ml-0.25 ml | 24 h | I + II | I + II |
| 15 | MTBE | Slurry | 1 g/5 ml-0.25 ml | 24 h | II | II |
| 16 | MTBE | Slurry | 1 g/5 ml | 48 h | — | I + II |
| 17 | Diethyl ether | Slurry | 1 g/5 ml | 24 h | II | II |
| 18 | Diethyl ether | Slurry | 1 g/5 ml | 48 h | — | II |

### Example 19

### Preparation of Amorphous Pantoprazole

Pantoprazole sodium (5.0 g) was dissolved in 25 ml of water (25 ml). Dichloromethane (25 ml) was added to the solution. Acetic acid (7 ml) was added dropwise to the stirred mixture. After phase separation dichloromethane was evaporated in vacuum evaporator at 30° C., giving after drying (vacuum 20 mm, 40° C., 1 hr) amorphous pantoprazole.

Having thus described the invention with respect to certain preferred embodiments and further illustrated it with examples, those skilled in the art may come to appreciate substitutions and equivalents that albeit not expressly described are taught and inspired by this invention. Whereas such substitutions and equivalents do not depart from the spirit of the invention, they are within its scope which is defined by the claims that follow.

### Example 20

### Preparation of Pantoprazole Sodium

Pantoprazole (3.0 g, 7.8 mM) was stirred with ethyl acetate (30 ml) and 47% aqueous sodium hydroxide (0.7 g, 7.8 mM) was added, and the mixture was stirred overnight at room temperature. The solid was filtered and giving after drying pantoprazole sodium sesquihydrate (3.2 g, 96%).

What is claimed is:

1. Crystalline solid pantoprazole characterized by a powder X-ray diffraction pattern having peaks at 6.6, 13.2, 13.7, 15.7, 23.1, and 23.4±0.2 °2θ.

2. The crystalline solid pantoprazole of claim 1 further having peaks in the powder X-ray diffraction pattern at 20.1, 20.9, 25.9, 27.5, 29.1±0.2 °2θ.

3. The crystalline solid pantoprazole of claim 1 having a powder X-ray diffraction pattern substantially as depicted in FIG. 1.

4. The crystalline solid pantoprazole of claim 1 having an infrared spectrum with bands at 1385, 1264, 1244, 1180, and 1027 at cm⁻¹.

5. The crystalline solid pantoprazole of claim 4 having an infrared spectrum substantially as depicted in FIG. 2.

6. A process for preparing the crystalline solid pantoprazole of claim 1, comprising
a) dissolving pantoprazole in a solvent;
b) precipitating crystals of the pantoprazole of claim 1 from the solution; and
c) separating the crystals from the solvent.

7. The process of claim 6, wherein the solvent is selected from the group consisting of ethanol, n-propanol, and acetone.

8. The process of claim 6, wherein the solution is heated before precipitating the crystals.

9. The process of claim 8, wherein the solution is then cooled to precipitate the crystals.

10. A process for preparing the crystalline solid pantoprazole of claim 1, comprising
a) forming a slurry of amorphous pantoprazole in a diluent;
b) maintaining the slurry for a period of time sufficient to convert the amorphous pantoprazole to the pantoprazole of claim 1; and
c) separating the pantoprazole of claim 1 from the diluent.

11. The process of claim 10, wherein the slurry is maintained for at least twelve hours.

12. The process of claim 10, wherein the diluent is selected from the group consisting of ethanol, acetone, n-propanol, ethyl acetate, tetrahydrofuran, sec-butanol, dimethylcarbonate, mixtures of methyl tert butyl ether and water, mixtures of dimethylcarbonate and water, mixtures of sec butanol and water, and mixtures of dichloromethane and water.

13. Crystalline solid pantoprazole characterized by a powder X-ray diffraction pattern having peaks at 5.8, 7.5, 9.3. 15.0, 22.0, and 22.6±0.2 °2θ.

14. The crystalline solid pantoprazole of claim 13 further having peaks in the powder X-ray diffraction pattern at 17.3, 18.6, 19.4, 20.8, 24.0, 24.8, and 25.5±0.2 °2θ.

15. The crystalline solid pantoprazole of claim 13 having a powder X-ray diffraction pattern substantially as depicted in FIG. 3.

16. The crystalline solid pantoprazole of claim 13 having an infrared spectrum having bands at 3195, 1196, and 1584 at cm⁻¹.

17. The crystalline solid pantoprazole of claim 16 having an infrared spectrum substantially as depicted in FIG. 4.

18. The crystalline solid pantoprazole of claim 13 having a melting endotherm at about 143° C. to about 146° C.

19. A process for preparing the crystalline solid pantoprazole of claim 13, comprising
a) forming a slurry of amorphous pantoprazole in a diluent;
b) maintaining the slurry for a period of time sufficient to convert the amorphous pantoprazole to the pantoprazole of claim 13; and
c) separating the pantoprazole of claim 13 from the diluent.

20. The process of claim 19, wherein the slurry is maintained for about twenty-four hours.

21. The process of claim 19, wherein the solvent is selected from the group consisting of diethyl ether and tert-butyl methyl ether.

22. A process for preparing a mixture of the crystalline solids pantoprazole of claims 1 and 13 comprising:
a) forming a slurry of amorphous pantoprazole in a diluent;
b) maintaining the slurry for a period of time sufficient to convert the amorphous pantoprazole to the pantoprazole of claims 1 and 13;
c) separating the pantoprazole of claims 1 and 13 from the diluent.

24. The process of claim 22, wherein the slurry is maintained for at least about twenty-four hours.

25. The process of claim 22, wherein the diluent is selected from the group consisting of mixtures of toluene and water, and methyl tert butyl ether.

26. Amorphous pantoprazole.

27. A process for preparing the amorphous pantoprazole of claim 26, comprising
a) partitioning pantoprazole between the organic and aqueous phases of a biphasic mixture of a water-immiscible organic liquid and water;
b) adding acid to the mixture;
c) separating the organic phase and the water; and
d) recovering amorphous pantoprazole from the organic phase.

28. The process of claim 27, wherein pantoprazole is partitioned by adding pantoprazole sodium to the mixture.

29. The process of claim 27, wherein the amorphous pantoprazole is recovered by evaporating the organic liquid.

30. The process of claim 27, wherein the organic liquid is dichloromethane.

31. The process of claim 27, wherein the acid is acetic acid.

32. A process for preparing a salt of pantoprazole comprising converting the pantoprazole of any of claims 1, 13, and 26 to a salt of pantoprazole.

33. A salt of pantoprazole prepared by the process of claim 32.

34. The process of claim 32, wherein the salt is pantoprazole sodium.

35. Pantoprazole sodium prepared by the process of claim 34.

36. A process for preparing a salt of pantoprazole comprising:
a) stirring pantoprazole of any of claims 1, 13, and 26 with ethyl acetate and aqueous sodium hydroxide; and
b) isolating pantoprazole sodium.

37. Pantoprazole sodium prepared by the process of claim 36.

38. The product of claim 37, wherein the pantoprazole sodium is sesquihydrate.

39. The process of claim 36, wherein the pantoprazole is the pantoprazole of claim 1.

40. The process of claim 36, wherein the mixture is stirred overnight at room temperature.

41. A pharmaceutical composition comprising the pantoprazole of any of claims 1, 13, and 26 and a pharmaceutical excipient.

42. The pharmaceutical composition of claim 41 that is a solid.

43. The pharmaceutical composition of claim 42 that is a tablet.

44. The pharmaceutical composition of claim 41 that is a liquid.

45. A method of inhibiting gastric acid secretion in the stomach of a patient comprising administering to the patient the pantoprazole of any of claims 1, 13, and 26.

46. A pharmaceutical composition comprising the pantoprazole of any of claims 33, 37 and 38 and a pharmaceutical excipient.

47. The pharmaceutical composition of claim 46 that is a solid.

48. The pharmaceutical composition of claim 47 that is a tablet.

49. The pharmaceutical composition of claim 46 that is a liquid.

50. A method of inhibiting gastric acid secretion in the stomach of a patient comprising administering to the patient the pantoprazole of any of claims 33, 37, and 38.
